(19) United States
(12) Patent Application Publication
(10) Pub. No.: US 2005/0019804 A1
(43) Pub. Date: Jan. 27, 2005

(54) Random array of microspheres

(75) Inventors: Krishnan Chari, Fairport, NY (US); Zhanjun Gao, Rochester, NY (US); Joseph S. Sedita, Albion, NY (US); Ramasubramaniam Hanumanthu, Baltimore, MD (US); Charles P. Lusignan, Rochester, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/868,082

(22) Filed: Jun. 15, 2004

Related U.S. Application Data
(63) Continuation-in-part of application No. 10/625,428, filed on Jul. 23, 2003.

(51) Int. Cl. C12Q 1/68; C12M 1/34; B05D 3/00

(52) U.S. Cl. 435/6; 435/287.2; 427/2.11

(57) ABSTRACT

An element containing an array of microspheres on a support is described, and a method of making the element, wherein the method includes coating a support with a coating composition to form a receiving layer with a modifiable elastic modulus; coating on the receiving layer a dispersion of microspheres in a fluid suspension; modifying the modulus of the receiving layer to allow the microspheres to partially submerge into the receiving layer; removing the fluid suspension from the receiving layer; and fixing the microspheres in the receiving layer so that the element can withstand wet processing.

### CROSS REFERENCE TO RELATED APPLICATIONS

This application is a continuation-in-part of U.S. application Ser. No. 10/625,428 filed Jul. 23, 2003, which relates to commonly assigned copending application Ser. No. 09/942,241, filed Aug. 29, 2001, entitled “RANDOM ARRAY OF MICROSPHERES.” The copending application is incorporated by reference herein for all that it contains.

### FIELD OF THE INVENTION

The present invention concerns biological or sensor micro-array technology in general. In particular, it concerns a micro-array coated on a substrate that contains no sites designated to attract the microspheres prior to coating.

### BACKGROUND OF THE INVENTION

Ever since they were invented in the early 1990s, high-density arrays formed by spatially addressable synthesis of bioactive probes on a two-dimensional solid support have greatly enhanced and simplified the process of biological research and development (see Science, 251, 767-773, 1991). The key to current micro-array technology is deposition of a bioactive agent at a single spot on a microchip in a “spatially addressable” manner.

Current technologies have used various approaches to fabricate micro-arrays. For example, U.S. Pat. Nos. 5,412,087, and 5,489,678 demonstrate the use of a photolithographic process for making peptide and DNA micro-arrays. The patents teach the use of photolabile protecting groups to prepare peptide and DNA micro-arrays through successive cycles of deprotecting a defined spot on a 1 cm×1 cm chip by photolithography, then flooding the entire surface with an activated amino acid or DNA base. Repetition of this process allows construction of a peptide or DNA micro-array with thousands of arbitrarily different peptides or oligonucleotide sequences at different spots on the array. This method is expensive. An ink jet approach is being used by others (e.g., U.S. Pat. Nos. 6,079,283; 6,083,762; and 6,094,966) to fabricate spatially addressable arrays, but this technique also suffers from high manufacturing cost in addition to the relatively large spot size of 40 to 100 μm. Because the number of bioactive probes to be placed on a single chip usually runs anywhere from 1,000 to 100,000 probes, the spatial addressing method is intrinsically expensive regardless of how the chip is manufactured.

An alternative approach to the spatially addressable method is the concept of using fluorescent dye-incorporated polymeric beads to produce biological multiplexed arrays. U.S. Pat. No. 5,981,180 discloses a method of using color-coded beads in conjunction with flow cytometry to perform multiplexed biological assays. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere at a time through a flow cytometry cell to decode sample information. U.S. Pat. No. 6,023,540 discloses the use of fiber-optic bundles with pre-etched microwells at distal ends to assemble dye-loaded microspheres. A unique bioactive agent was attached to the surface of each spectrally addressed microsphere, and thousands of microspheres carrying different bioactive probes combined to form an array of beads on pre-etched microwells of fiber optical bundles.

More recently, a novel optically encoded microsphere approach was accomplished by using different sized zinc sulfide-capped cadmium selenide nanocrystals incorporated into microspheres (Nature Biotech., 19, 631-635, (2001)). Given the narrow band width demonstrated by these nanocrystals, this approach significantly expands the spectral barcoding capacity in microspheres.

Even though the “spectrally addressed microsphere” approach does provide an advantage in terms of its simplicity over the old fashioned “spatially addressable” approach in micro-array making, there are still needs in the art to make the manufacture of biological micro-arrays less difficult and less expensive.

U.S. Ser. No. 09/942,241, “Random Array of Microspheres,” filed Aug. 29, 2001, teaches various coating methods and exemplifies machine coating, whereby a support is coated with a fluid coating composition comprising microspheres dispersed in gelatin, as shown in FIGS. 1a and 1b. Immediately after coating, the support is passed through a chill-set chamber in the coating machine where the gelatin undergoes rapid gelation and the microspheres are immobilized, as shown in FIG. 1c. The excess fluid is removed by evaporation, as shown in FIG. 1d. While this process provides a huge manufacturing advantage over then existing technologies, the process needs some refinement in order to maximize its full potential value to the art. The problem is that during such machine coating and rapid gelation, the gelling agent tends to cover the surface of the microspheres as shown in FIG. 1e, thereby preventing the analyte (such as DNA) from penetrating through the gel overcoat and hybridizing with probes on the surface of the microspheres.

U.S. Ser. No. 10/062,326, “Method of Making Random Array of Microspheres Using Enzyme,” filed Jan. 31, 2002, overcomes the problem outlined above by enzymatically removing the gelling agent from the surface of the microspheres without damaging their integrity or the DNA probes on their surfaces. The enzyme-treated surface maintains its physical integrity through the entire DNA hybridization process and the micro-array shows a very strong hybridization signal. The advantage of U.S. Ser. No. 10/062,326 is that enzyme digestion can be easily controlled to remove the required amount from the gel overcoat. Further, the enzyme, a protease, is readily available and economical to obtain. However, there is a disadvantage in that an additional process (enzyme digestion) is required and this involves additional time and cost.

U.S. Ser. No. 10/092,803, “Random Array of Microspheres,” filed Mar. 7, 2002, describes a process of preparing a random bead micro-array by coating a suspension of microspheres, without gelling agent but containing a cross-linker for the gelling agent, onto a receiving layer capable of undergoing sol-gel transition, as shown in FIGS. 2a and 2b. The microspheres partially submerge into the receiving layer as shown in FIG. 2c, and the receiving layer is then cross-linked as shown in FIG. 2d. The excess fluid from the suspension is removed by evaporation, as shown in FIG. 2e, to form a micro-array. While this approach is an improvement over U.S. Ser. No. 09/942,241, it is not completely successful in preventing deposition of gelling agent onto the surfaces of the microspheres, as shown in FIG. 2f, because the gelling agent in the receiving layer can dissolve in the aqueous suspension used to deposit the microspheres, and can re-deposit onto the microspheres when the suspension is spread on the receiving layer. Furthermore, the presence of cross-linker in the suspension can cross-link biological molecules on the surfaces of the microspheres and render them ineffective as probes.

A method is needed wherein a suspension of microspheres can be spread onto a receiving layer wherein the material of the receiving layer does not dissolve in the suspension or medium in which the microspheres are being transported. Furthermore, the composition of the receiving layer has to be such so as to permit sufficient submerging of the microspheres in the receiving layer to prevent lateral aggregation when the solvent in the suspension is removed, such as by evaporation.

### SUMMARY OF THE INVENTION

The present invention provides a method of making an element, for example, a micro-array, having microspheres, the method comprising coating a support with a coating composition to form a receiving layer, said layer having a modulus that can be modified by crosslinking; allowing partial cross-linking of the receiving layer to achieve an elastic modulus that permits partial submerging of the microspheres into the partially cross-linked receiving layer; coating on the partially cross-linked receiving layer a dispersion of microspheres in a fluid suspension, each microsphere having a position; allowing the microspheres to partially submerge into the partially cross-linked receiving layer; removing the fluid suspension from the partially cross-linked receiving layer; and allowing the partially cross-linked receiving layer to further cross-link so that the microspheres maintain their respective positions during and after wet processing.

In another embodiment of the invention, there is disclosed an element comprising a support; a water-insoluble receiving layer on the support, wherein the receiving layer comprises a receiving material; and randomly-spaced microspheres fixed and partially submerged in the receiving layer, wherein the microspheres have surfaces exposed above the receiving layer, each exposed surface having at least one probe attached for interacting with an analyte, and wherein the exposed surfaces of the microspheres are free of receiving layer material.

The receiving layer and the support are characterized by an absence of sites designed to specifically interact physically or chemically with the microspheres. Hence, the distribution of the microspheres is not predetermined or directed, but is entirely random.

### ADVANTAGES

The invention utilizes a unique coating technology to prepare a micro-array on a substrate that need not be pre-etched with microwells or premarked in any way with sites to attract the microspheres, as disclosed in the art. By using unmarked substrates or substrates that need no pre-coating preparation, the present invention provides a huge manufacturing advantage compared to the existing technologies. The invention discloses a method whereby addressable mixed microspheres in a dispersion are randomly distributed on a receiving layer that has no wells or sites to attract the microspheres.

The present invention provides a micro-array that is less costly and easier to prepare than those previously disclosed because the substrate does not have to be modified; nevertheless the microspheres remain immobilized on the substrate.

Further, the present invention provides a micro-array wherein, in contrast to U.S. Ser. No. 09/942,241, filed Aug. 29, 2001, the microsphere surfaces are exposed but without employing the additional process step (enzyme digestion) disclosed in U.S. Ser. No. 10/062,326, filed Jun. 3, 2002. Exposed microsphere surfaces facilitate easier access of the analyte to probes attached to the surfaces of the microspheres. By “analyte” is meant molecules with functionalities capable of interacting chemically or physically with specific moieties on the microsphere surface, herein called “probes”. In the present invention, the analyte is preferably a nucleic acid or protein.

One of the key elements of the present invention is the selection of the receiving layer. The receiving layer must have a desired physical property that allows the microspheres to sufficiently submerge in the receiving layer, thereby preventing lateral aggregation. Specific requirements on the physical properties of the receiving layer will be discussed in detail later.

### BRIEF DESCRIPTION OF THE DRAWINGS

FIGS. 1a to 1e are schematics showing one method employed in the prior art for preparing a microsphere micro-array. FIG. 1a shows any suitable support 1; FIG. 1b shows a fluid layer 2 containing microspheres (beads) 3, gelling agent and a chemical cross-linking agent spread over the support of FIG. 1a; FIG. 1c shows the fluid layer wherein the gelling agent has undergone sol-gel transition thereby immobilizing the microspheres 3 in gel 4; FIG. 1d shows micro-array 5 formed by the evaporation of excess fluid 2 from the gel layer 4; and FIG. 1e shows the crosslinked fluid layer 6 which permanently fixes the microspheres 3 in the micro-array, leaving a film 7 of gelling agent on the surfaces of the microspheres 3.

FIGS. 2a to 2f are schematics of another prior art process of preparing a random microsphere micro-array wherein FIG. 2a shows any suitable support 1; FIG. 2b shows the support 1 coated with non-cross-linked gelling agent 8; FIG. 2c shows a fluid layer 2 carrying microspheres 3 bearing probes, and a cross-linker for the gelling agent 8, disposed on the support 1 of FIG. 2b; FIG. 2d shows the microspheres 3 of FIG. 2c sunk into the non-cross-linked gelling agent 8. As seen in FIG. 2e, the layer with the gelling agent 8 undergoes sol-gel transition to a gel 4 and thereby immobilizes the microspheres 3. FIG. 2e shows the evaporation of fluid from the fluid layer 2; FIG. 2f shows the final micro-array 5 wherein the microspheres 3 still have a coating of gel 4 on their surfaces because of dissolution of gelling agent 8 into the fluid layer 2.

FIGS. 3a to 3g are schematics of one embodiment of the present invention wherein FIG. 3a shows any suitable support 1; FIG. 3b shows a cross-linkable composition and chemical cross-linking agent spread over the support 1 of FIG. 3a to form a receiving layer 9; FIG. 3c shows the partially cross-linked receiving layer 10 the elastic modulus of which is adjusted to permit indentation by microspheres in a fluid suspension that will be spread over it; FIG. 3d shows a fluid suspension 11 containing microspheres 3 spread over the partially cross-linked receiving layer 10 of FIG. 3c; FIG. 3e shows the microspheres 3 partially sinking into the partially cross-linked receiving layer 10; FIG. 3f shows the evaporation of fluid suspension 11 to expose the surfaces of the microspheres 3; FIG. 3g shows a further chemically cross-linked receiving layer 12 that makes the micro-array 5 robust to wet processing.

FIGS. 4a to 4g are schematics of another embodiment of the present invention wherein FIG. 4a shows any suitable support 1; FIG. 4b shows a cross-linkable composition spread over the support 1 of FIG. 4a to form a receiving layer 9; FIG. 4c shows the partially cross-linked receiving layer 10 cross-linked by radiation 13 such as ultra-violet (UV) radiation, ionizing radiation or electron beam irradiation, to an elastic modulus sufficient to permit indentation by microspheres in a fluid suspension that will be spread over it; FIG. 4d shows a fluid suspension 11 containing microspheres 3 spread over the partially cross-linked receiving layer 10 of FIG. 4c; FIG. 4e shows the microspheres 3 partially sinking into the partially cross-linked receiving layer 10; FIG. 4f shows the evaporation of fluid suspension 11 to expose the surfaces of the microspheres 3; FIG. 4g shows a further cross-linked receiving layer 12 cross-linked by radiation 13 such as UV radiation, ionizing radiation or electron beam irradiation to make the micro-array 5 robust to wet processing.

FIG. 5 is yet another schematic of a process of the invention wherein FIG. 5a shows any suitable support 1; FIG. 5b shows a fluid containing a gelling agent and a slow acting chemical cross-linking agent for the gelling agent spread over the support of FIG. 5a to form a receiving layer 9; FIG. 5c shows the sol-gel transitioned receiving layer 14 wherein the gelling agent has gelled to have an elastic modulus sufficient to permit indentation by the microspheres 3; FIG. 5d shows a fluid suspension 11 containing microspheres 3 at a temperature below the sol-gel transition of the gelling agent in the receiving layer spread over the gelled receiving layer 14 of FIG. 5c; FIG. 5e shows the microspheres 3 partially sinking into the gelled receiving layer 14; FIG. 5f shows the evaporation of fluid suspension 11 to expose the surfaces of the microspheres 3; FIG. 5g shows the chemically cross-linked receiving layer 12 which makes the micro-array 5 robust to wet processing.

FIG. 6 is a diagram of a 1 cm² area with 1000 microspheres, wherein no two microspheres overlap.

FIG. 7 is a plot of the data in Table 1, showing distribution of nearest neighbor separation distances between microspheres of FIG. 6.

FIG. 8 is schematic showing the forces on a microsphere. FIG. 9 is a plot showing upper and lower bounds of a feasible modulus for a 10 μm microsphere wherein L is 30 μm.

FIG. 10 is a plot showing upper and lower bounds of a feasible modulus for a 5 μm microsphere wherein L is 30 μm.

FIG. 11 is a plot showing upper and lower bounds of a feasible modulus for a 15 μm microsphere wherein L is 20 μm.

FIG. 12 is a plot showing upper and lower bounds of a feasible modulus for a 20 μm microsphere wherein L is 20 μm.

FIG. 13 is a plot showing upper and lower bounds of a feasible modulus for a 10 μm microsphere wherein L is 5 μm.

FIG. 14 is a plot showing upper and lower bounds of a feasible modulus for a 20 μm microsphere wherein L is 2.5 μm.

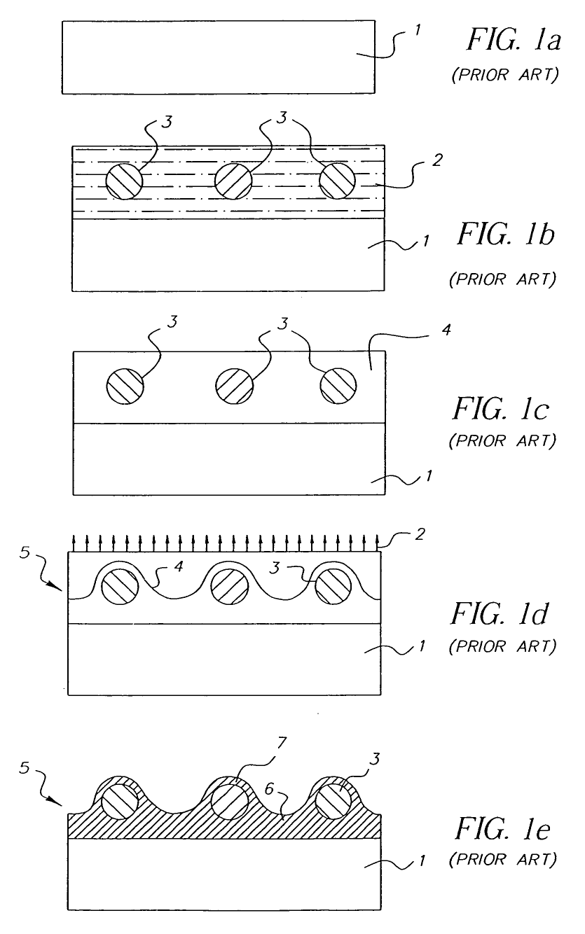

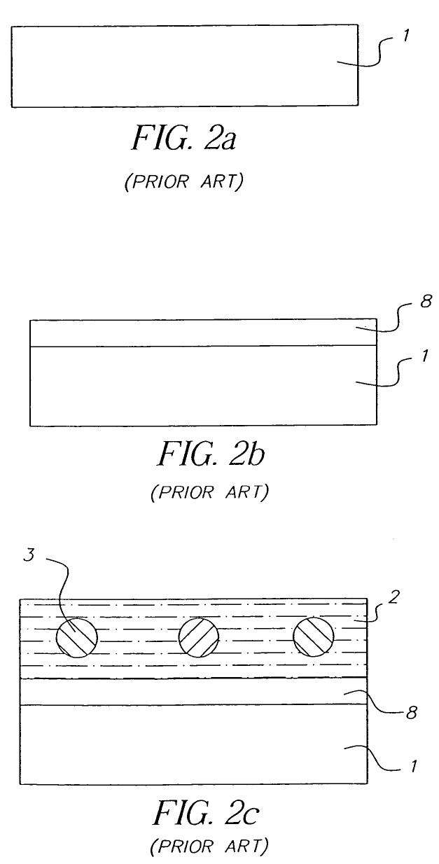

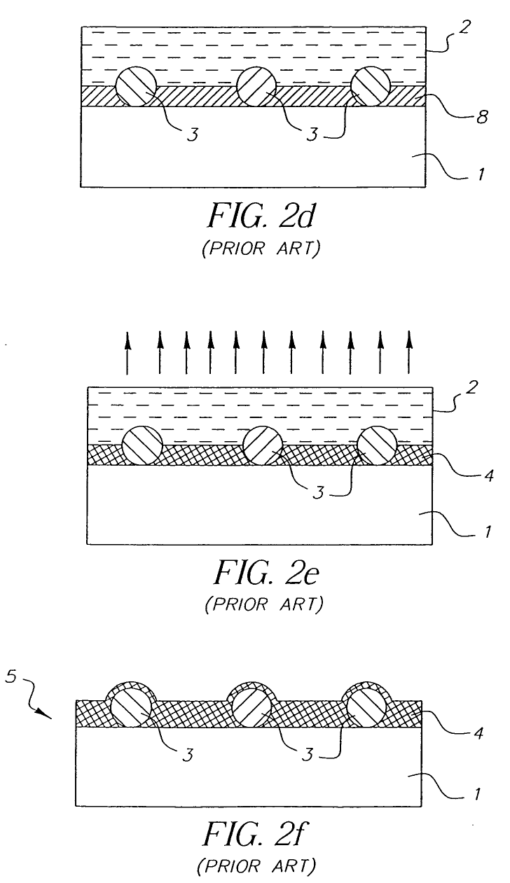

### DETAILED DESCRIPTION OF THE INVENTION

As used herein, the term “sol-to-gel transition” or “gelation” means a process by which fluid solutions or suspensions of particles form continuous three-dimensional networks that exhibit no steady state flow. This can occur in polymers by polymerization in the presence of polyfunctional monomers; by covalent cross-linking of a dissolved polymer that possesses reactive side chains; and by secondary bonding, for example, hydrogen bonding, between polymer molecules in solution. Polymers such as gelatin exhibit thermal gelation that is of the last type. The process of gelation, or setting, is characterized by a discontinuous rise in viscosity. See, P. I. Rose, “The Theory of the Photographic Process,” 4^{th} Edition, T. H. James ed., pages 51 to 67.

As used herein, the term “gelling agent” means a substance that can undergo gelation as described above. Examples include materials that undergo thermal gelation, such as gelatin, water-soluble cellulose ethers, or poly(n-isopropylacrylamide), or substances that may be chemically cross-linked by a borate compound, such as poly(vinyl alcohol). Other gelling agents include polymers that may be cross-linked by radiation such as ultraviolet radiation, ionizing radiation, or electron beam radiation. Examples of gelling agents include acacia, alginic acid, bentonite, carbomer, carboxymethylcellulose sodium, cetostearyl alcohol, colloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alcohol, povidone, propylene glycol alginate, sodium alginate, sodium starch glycolate, starch, tragacanth and xanthum gum. Other gelling agents known in the art, such as those set forth in Secundum Artem, Vol. 4, No. 5, Lloyd V. Allen, can also be used. A preferred gelling agent is alkali-pretreated gelatin.

As used herein, the term “random distribution” means a spatial distribution of elements showing no preference or bias. Randomness can be measured in terms of compliance with that which is expected by a Poisson distribution.

The present invention teaches a method for making a random array of microspheres, also referred to as “beads,” on a substrate that can include a receiving layer. The microspheres are deposited on the receiving layer in such a way that a portion of the surface of each microsphere is exposed above the receiving layer. The distribution or pattern of the microspheres on the receiving layer or substrate is entirely random and the microspheres are not attracted or held to sites that are pre-marked or predetermined on the receiving layer or substrate as in other methods previously known in the art.

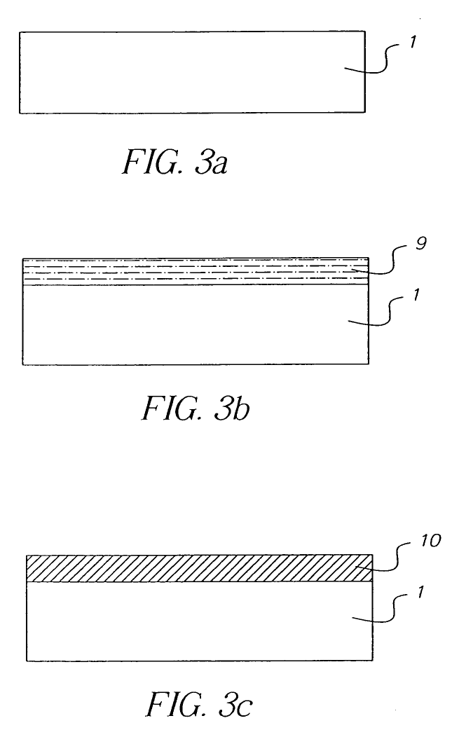

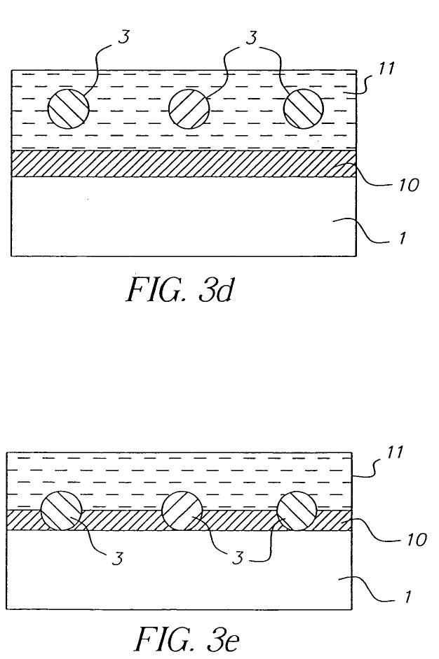

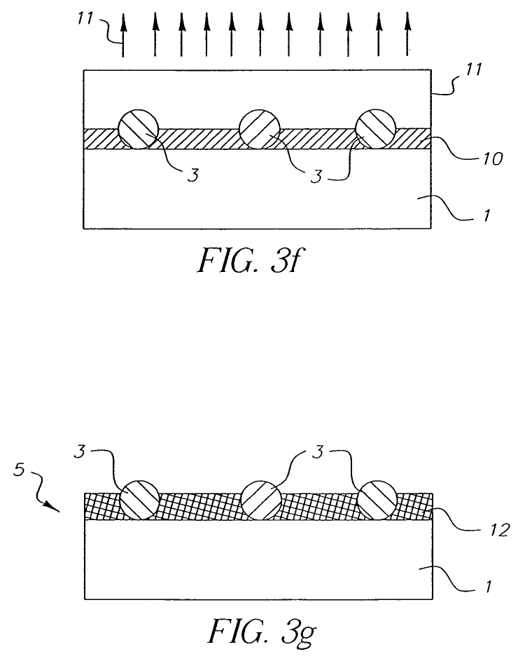

The random array is achieved by first coating on any suitable surface or support 1 (FIGS. 3a, 4a, 5a) a fluid layer containing a gelling agent and a chemical cross-linker for the gelling agent, forming a receiving layer 9 (FIGS. 3b, 4b, 5b). The support 1 can be, for example, glass, paper, metal, a polymeric material, a composite material, or a combination thereof, so long as the support provides a surface on which a receiving layer can be formed. The gelling agent in the receiving layer 9 is allowed to partially cross-link to form a partially cross-linked receiving layer 10 (FIGS. 3c, 4c, 5c). A fluid suspension 11 of microspheres 3 is then spread over the partially cross-linked receiving layer 10 (FIGS. 3d, 4d, 5d). The partially cross-linked receiving layer 10 is insoluble in the fluid suspension 11. The microspheres 3 settle as least partially into the partially cross-linked receiving layer 10 (FIGS. 3e, 4e, 5e). The extent of settling is related to the elastic modulus of the partially cross-linked receiving layer 10, the interfacial surface energy of the material of the microspheres 3, and the interfacial surface energy of the fluid suspension 11. The microspheres 3 can settle all or part-way into the partially cross-linked receiving layer 10. One or more microsphere can settle to the same depth in partially cross-linked receiving layer 10. According to certain embodiments, at least some of the microspheres 3 can settle to the bottom of partially cross-linked receiving layer 10, resting on support 1.

The elastic modulus of the partially cross-linked receiving layer 10 is controlled by the cross-link density of the partially cross-linked receiving layer 10, defined as the moles of cross-linked material per unit volume. The cross-link density is in turn related to one or more of the concentration of chemical cross-linking agent; the duration of chemical cross-linking; the intensity and time of radiation, such as UV, ionizing, or electron beam radiation; and the type of cross-linking employed.

Alternatively, it is possible to use physical gelation instead of chemical cross-linking or radiation induced cross-linking. Physical gelation is based on formation of hydrogen bonds in the receiving layer. The cross-link density in physical gelation is related to the concentration of gelling agent and the difference between the temperature of the partially cross-linked receiving layer and the gel point or sol-gel transition temperature of the gelling agent in the partially cross-linked receiving layer. In this case, the temperature of the fluid suspension at the time of coating is maintained below the sol-gel transition temperature of the gelling agent in the partially cross-linked receiving layer to prevent dissolution of the gelling agent in the partially cross-linked receiving layer into the fluid suspension.

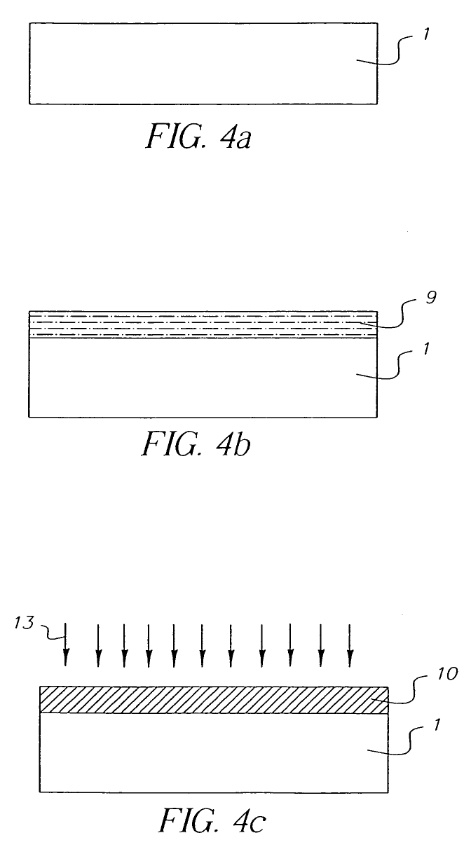

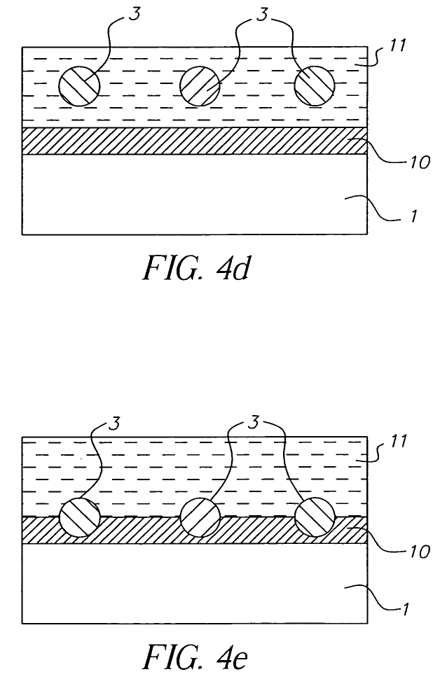

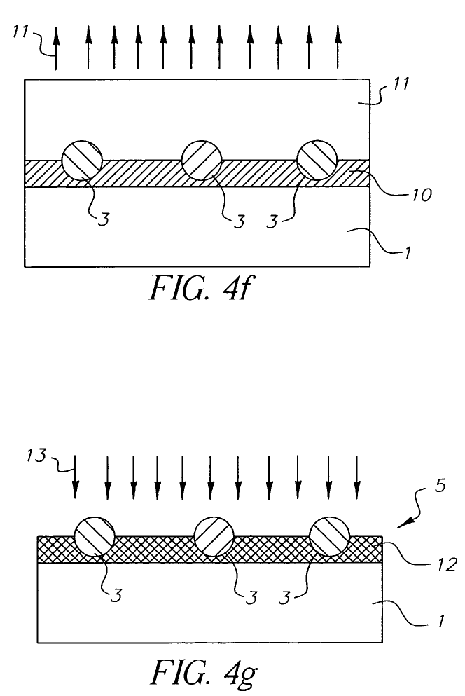

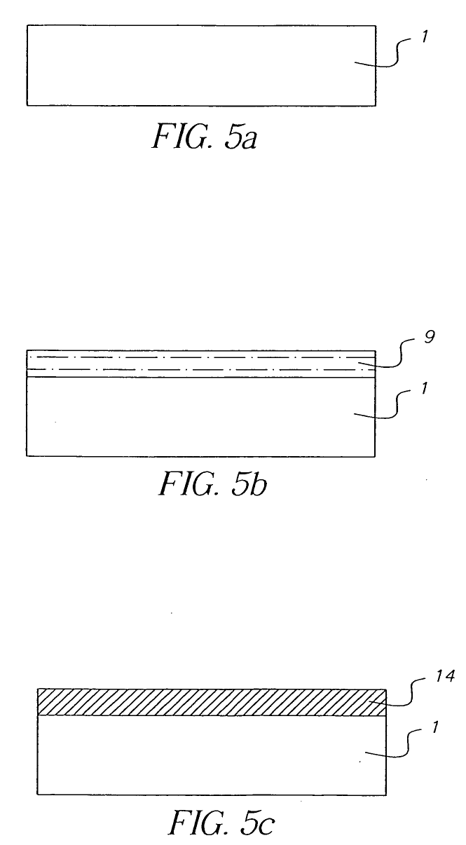

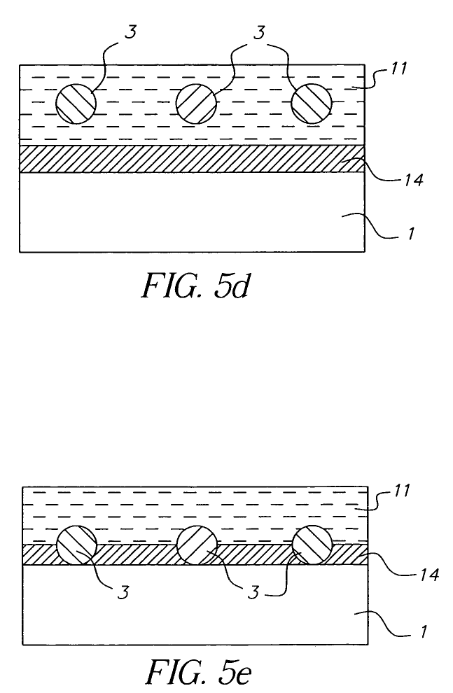

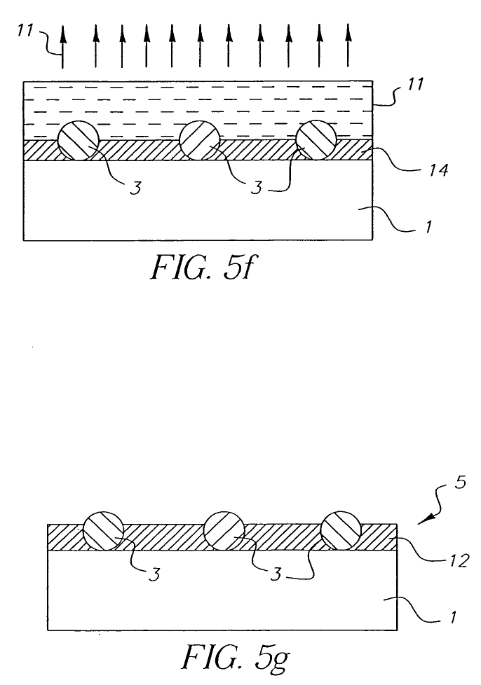

Evaporation of fluid suspension 11 may be achieved by blowing air over the fluid suspension 11, heating the fluid suspension 11, or a combination thereof, to evaporate the fluid (FIGS. 3f, 4f, 5f), leaving an array 5. After the array 5 has been fully fabricated, the cross-linking reaction of the partially cross-linked receiving layer 10 containing a crosslinked gelling agent initiated earlier by addition of the cross-linker may go to completion to permanently fix the microspheres 3 in place in a cross-linked receiving layer 12 (FIGS. 3g, 5g). If gelatin is used as the gelling agent, preferred cross-linkers may be compounds such as bis(vinylsulfone)methane, glutaraldehyde or succinaldehyde. Alternatively, as shown in FIG. 4g, additional radiation 13, such as UV radiation, ionizing radiation, or electron beam irradiation, may be used to effect additional cross-linking. The cross-linked receiving layer is insoluble, allowing wet-processing of the formed micro-array without dissolution or degradation of the cross-linked receiving layer.

As shown in FIGS. 3g, 4g, and 5g, the microspheres 3 in the array 5 have no receiving layer material attached to or covering the exposed surfaces of the microspheres 3. This enables attachment of functionalized chemical or biological groups, probes, and analytes to the exposed surfaces of the microspheres.

The above described methods of preparing a partially cross-linked receiving layer by physical gelation, chemical cross-linking, or radiation, are designed to yield a partially cross-linked receiving layer capable of receiving the microspheres that has proper physical properties to ensure that no lateral aggregation of microspheres will occur during evaporation of fluid suspension from the partially cross-linked receiving layer to form the array. Two factors are important in determining if lateral aggregation of the microspheres will occur. One is capillary forces that drive the microspheres toward each other, as described in “Patterned Colloidal Deposition Controlled by Electrostatic and Capillary Forces,” J. Aizenberg, P. Braun, and P. Wiltzius, Physical Review Letters, Vol. 84, No. 13, 2000. The other is the degree of indentation of the microspheres into the partially cross-linked receiving layer. Capillary forces on the microspheres are proportional to the interfacial surface energy between the fluid suspension and the microspheres. At the stage of fluid suspension evaporation, when the combined thickness of the fluid suspension and the partially cross-linked receiving layer becomes comparable to the microsphere size, the capillary forces tend to cause lateral aggregation of microspheres in the partially cross-linked receiving layer. On the other hand, the surface force between the microspheres and the partially cross-linked receiving layer can cause the microsphere to indent into the relatively soft partially cross-linked receiving layer, as explained in “Surface Energy and the Contact of Elastic Solids,” K. Johnson et al., Proc. R. Soc. Lond., A. 324, 1971, permitting sufficient submerging of the microspheres into the partially cross-linked receiving layer to prevent lateral aggregation when the fluid suspension is removed by evaporation.

From the above discussion, it is easy to see that to prevent lateral aggregation, the physical property of the partially cross-linked receiving layer has to satisfy certain conditions. If the partially cross-linked receiving layer is hard, there is very little submerging of the microsphere into the partially cross-linked receiving layer, and lateral aggregation of the microspheres is likely to occur. On the other hand, if the partially cross-linked receiving layer is too soft, it will offer little resistance to the capillary forces driving lateral aggregation of the microspheres. The property of the partially cross-linked receiving layer to resist deformation can be represented by Young's modulus. A lower bound and an upper bound of the Young's modulus of the partially cross-linked receiving layer can be determined within which no lateral aggregation of the microspheres will occur. Methods of determining the bounds of the Young's modulus of the partially cross-linked receiving layer are provided in the example section herein.

The invention is a polymeric microsphere based random micro-array with each microsphere in the array having a distinct signature that distinguishes the microsphere from other microspheres in the micro-array. Such a signature may be based on color, shape, size of the microsphere, or a combination thereof. For signatures based on color, the color may be derived from mixing three dyes representing the primary colors, red, green and blue, to create thousands of distinguishable microspheres with distinct “color addresses” (unique RGB values, e.g. R=0, G=204, B=153). The microspheres can be made with sites on their surface that are “active”, meaning that at such sites physical or chemical interaction can occur between the microsphere and other molecules or compounds. Such compounds may be organic or inorganic. Examples of the molecule or compound include organic-nucleic acid, protein, or fragments thereof, or ionic compounds, including, for example, metal ions and salts. To the surface of each microsphere may be attached a pre-synthesized oligonucleotide, a monoclonal antibody, or any other biological or chemical agents. Therefore, each microsphere address, for example, a color, can correspond to a specific probe. These microspheres may be mixed in equal amounts, and the random micro-array fabricated by coating the mixed microspheres, for example, in a single layer.

Coating methods for coating a microsphere suspension are broadly described by Edward Cohen and Edgar B. Gutoff in Chapter 1 of “Modem Coating And Drying Technology”, Interfacial Engineering Series, v. 1, VCH Publishers Inc., New York, N.Y. (1992). Suitable coating methods may include knife coating, blade coating, dip coating, rod coating, air knife coating, gravure coating, forward and reverse roll coating, and slot and extrusion coating. Various coating aids as known in the art can be added to aid in coating the microsphere suspension on the substrate. For example, suitable coating aids can include surfactants, diluents, or thinning agents.

A biological sample that is fluorescently-labeled, chemiluminescently-labeled, or both, can be hybridized to the microsphere-based random micro-array. The signals from both addressable polymeric microspheres and biological samples non-selectively labeled with fluorescence, chemiluminescence, or both, may be analyzed with a charge-coupled device after image enlargement through an optical system. The recorded array image can be automatically analyzed by an image processing algorithm to obtain bioactive probe information based on the “address” of each microsphere, for example, the color code of each microsphere, and the information can be compared to the fluorescence/chemiluminescence image to detect and quantify specific biological analyte materials in the sample. Optical or other electro-magnetic means may be applied to ascertain signature.

Although microspheres or particles having a substantially curvilinear shape are preferred because of ease of preparation, particles of other shapes such as ellipsoidal or cubic particles may also be employed. Suitable methods for preparing the particles are known in the art, and can include emulsion polymerization as described, for example, in “Emulsion Polymerization” by I. Piirma, Academic Press, New York (1982), or limited coalescence, as described for example by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used. The formed microsphere can be colored using an insoluble colorant that is a pigment or dye that is not dissolved during coating or subsequent treatment. Suitable dyes may be oil-soluble in nature. It is preferred that the dyes are non-fluorescent when incorporated in the microspheres.

The microspheres are desirably formed to have a mean diameter in the range of 1 to 100 microns, for example, 1 to 50 microns, more preferably 3 to 30 microns, and most preferably 5 to 20 microns. It is preferred that the concentration of microspheres in the coating is in the range of 100 to a million per cm², more preferably 1000 to 200,000 per cm², and most preferably 10,000 to 100,000 per cm².

The microsphere can have chemical- or biological-functionalized groups attached to the surface of the microsphere to interact with a desired analyte. Methods of adding chemical or biological functional groups are known in the art.

The attachment of bioactive agents to the surface of chemically functionalized microspheres can be performed according to the published procedures in the art, for example, as set forth in Bangs Laboratories, Inc. Technote 205, Rev. 003, 30 Mar. 2002 (Bangs Laboratories, Inc., Fishers, Ind.). Some commonly used chemical functional groups include, but are not limited to, carboxyl, amino, hydroxyl, hydrazide, amide, chloromethyl, epoxy, aldehyde, etc. Examples of bioactive agents include, but are not limited to, oligonucleotides, DNA and DNA fragments, peptide nucleic acids (PNAs), peptides, antibodies, enzymes, proteins, and synthetic molecules having biological activities.

Methods of determining the Young's modulus range for the receiving layer for a given microsphere composition are set forth below.

### EXAMPLES

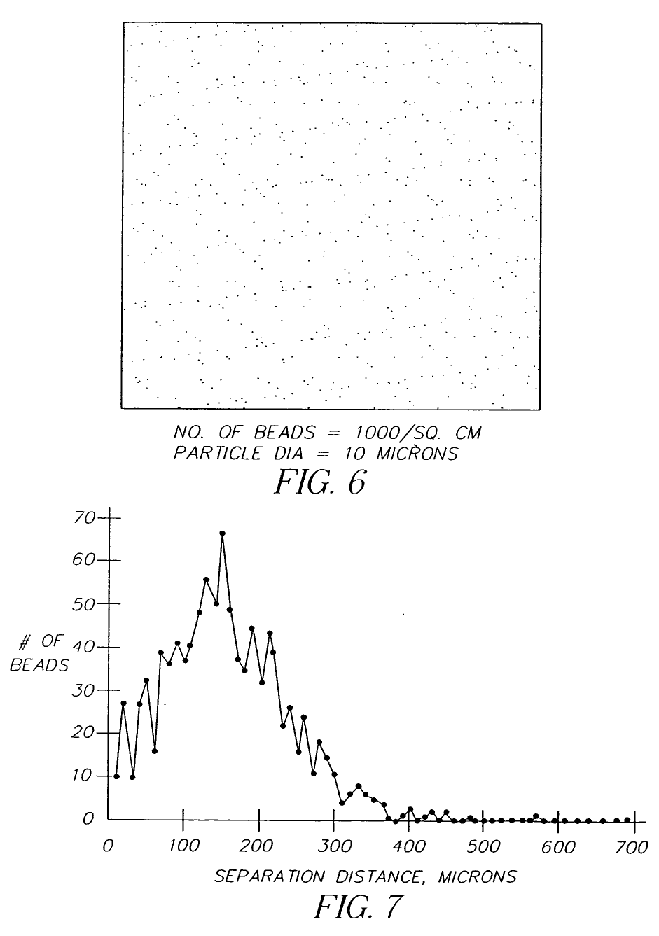

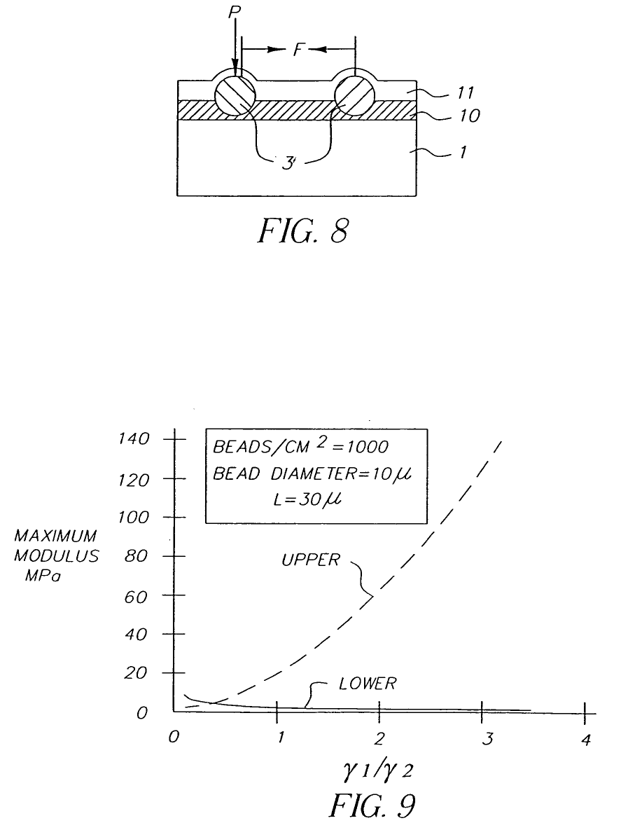

In the following example, Monte Carlo simulations as described in “Random Number Generation and Monte Carlo Methods (Statistics & Computing)” by James E. Gentle, Springer Verlag (1998), are performed to determine the distance between the microspheres that were introduced randomly. The results are then utilized in an analysis to calculate the Young's modulus of the receiving layer that avoids lateral aggregation of microspheres To simulate a random distribution of microspheres as achieved by the invention, 1000 microspheres of 10μ diameter were randomly dropped over a substrate with an area of 1 cm², such that no two of the microspheres overlapped, as shown in FIG. 6. The distribution of nearest neighbor separation distances between the microspheres in FIG. 6 is shown in Table 1 and is plotted in FIG. 7. The microspheres were randomly dropped on the substrate 20 times, and the average over all twenty simulations is shown in Table 2. A cumulative average for each of the nearest neighbor separation distances, equal to the percentage of total number of microspheres separated by at least the separation distance, is provided in Table 2.

Table 2 indicates that for this example (1000 microspheres/cm²; 10μ diameter microspheres), 95% of the microspheres are separated from their nearest neighbors by at least 30μ. This distance (30μ for this case) is called “L” and is the minimum measured distance of separation between microspheres for at least 95% of the microspheres.

The simulation was repeated for several cases of microsphere density and microsphere diameter, and L for each case was determined as described above over 20 repeated simulations at each microsphere diameter/density combination. The results are summarized in Table 3.

Using the values from Table 3, one can determine the modulus requirement for the partially cross-linked receiving layer to anchor the microspheres without lateral aggregation. The vertical force P and the lateral force F that act on each microsphere and effect lateral aggregation are illustrated in FIG. 8. The vertical force P holds the microsphere in place by pushing it down into the receiving layer. P is determined by the radius R of the microsphere and the interfacial surface energy between the microsphere and the partially cross-linked receiving layer, denoted γ₁, as taught by K. L. Johnson et al. in Proc. R. Soc., London A324, 301(1971). The force P is determined by the formula:
P=6πRγ₁ (1)
The horizontal force F that acts on each microsphere to effect lateral aggregation by lateral movement of the microspheres is determined by the radius of the sphere, R, the distance between the microspheres, L, and the interfacial surface energy between the microsphere and the fluid suspension containing the microsphere, denoted γ₂, as taught by Aizenburg et al. in PHYS. REV. LTRS., Vol. 84, No. 13, (2000). The lateral force F is determined by the formula:

$$F = 3 ( \frac{{R}^{2}}{L} ) {γ}_{2} \qquad ( 2 )$$

It can be seen from Equations (1) and (2) that for a given radius of microsphere and distance L between microspheres, the surface energies γ₁ and γ₂ determine the amount of vertical and lateral forces acting on each microsphere.

The interrelationship of the force P, the force F, and the Young's modulus of the partially cross-linked receiving layer will determine whether the microsphere is sufficiently anchored in the partially cross-linked receiving layer to resist lateral aggregation. When the microspheres are coated in the fluid suspension on the partially cross-linked receiving layer, the microspheres sink through the fluid suspension onto the partially cross-linked receiving layer. Depending on the relationship between the vertical force P on the microsphere, and the Young's modulus of the partially cross-linked receiving layer, the microsphere will at least partially penetrate the partially cross-linked receiving layer. As the fluid suspension is removed by evaporation, and the fluid suspension level becomes less than the height of the microsphere above the partially cross-linked receiving layer, capillary forces will come into effect, causing lateral force F. In order to move laterally, the microsphere needs to deform or plow through the partially cross-linked receiving layer. This movement is resisted by the ability of the partially cross-linked receiving layer to resist deformation, and such resistance is represented by the Young's modulus of the partially cross-linked receiving layer. Material with a higher Young's modulus exhibits a higher resistance to deformation, holding the microspheres in position in the partially cross-linked receiving layer. If the Young's modulus of the partially cross-linked receiving layer is too low, the partially cross-linked receiving layer will be too fluid, allowing easy movement of the microspheres, which could lead to lateral aggregation. If the Young's modulus of the partially cross-linked receiving layer is too high, the microsphere will not be able to penetrate the partially cross-linked receiving layer sufficiently to resist lateral movement, allowing the microsphere to slide along the surface of the partially cross-linked receiving layer without deforming it.

To determine the range of the Young's modulus of the receiving layer that will avoid lateral aggregation, finite element analyses are conducted. In accordance with conventional finite element analysis techniques, a geometric representation of the microspheres and the receiving layer is created by dividing the microspheres and layers into discrete elements (also called mesh). For given vertical and lateral forces, the finite element analysis determines if, for a selected value of Young's modulus, the microspheres will remain stationary or move laterally to form aggregation. The analysis provides a lower range, or lower bound, which is the lowest modulus at which the microspheres will not move, and an upper range, or upper bound, which is the highest modulus at which the microspheres will not move. The results can be plotted as a function of the modulus versus the ratio γ₁/γ₂, as shown in FIG. 9.

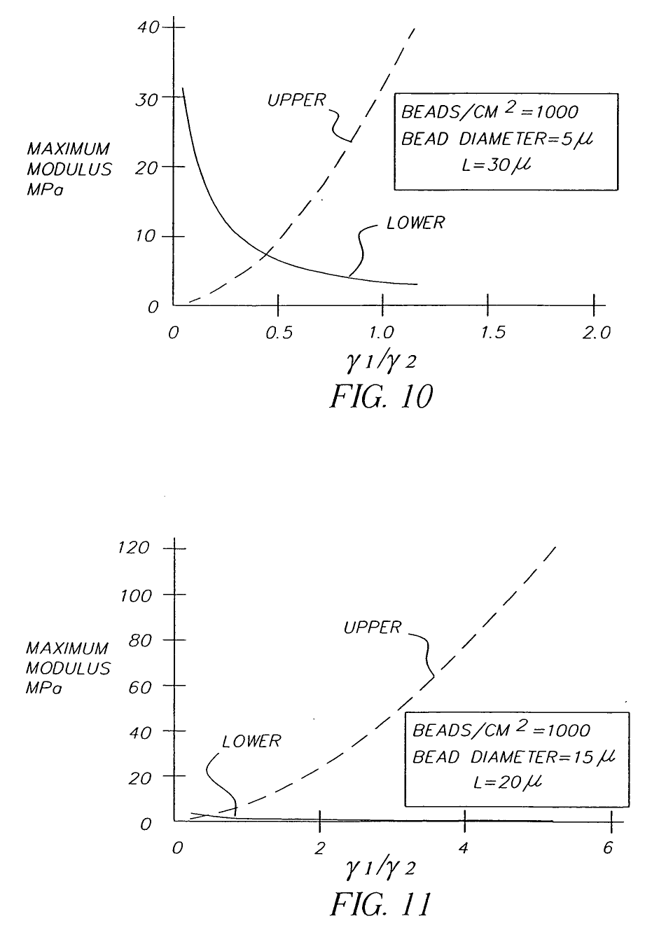

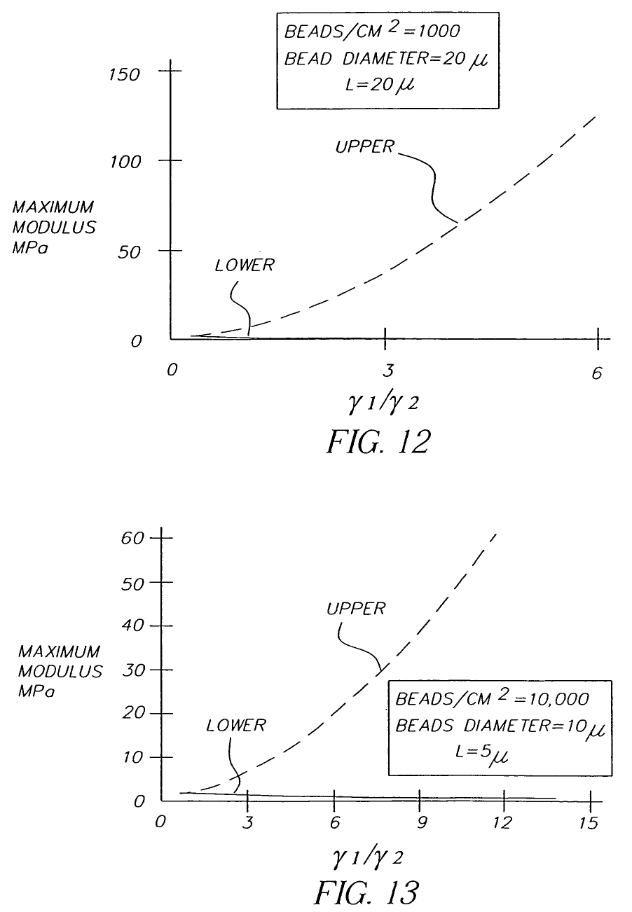

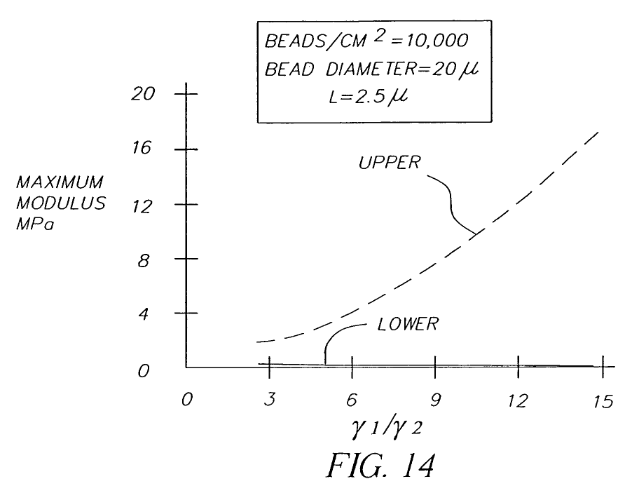

As shown in FIG. 9 (number of microspheres/cm\hat{ }2=1000, microsphere diameter=10μ, L=30μ), the desirable Young's modulus of the partially cross-linked receiving layer that prevents aggregation of the microspheres while keeping them in place is the region between the lower bound and the upper bound. The result depends on the magnitude of the interfacial surface energy between the microspheres and the partially cross-linked receiving layer, γ₁, and the interfacial surface energy between the microspheres and the fluid suspension, γ₂. The interfacial surface energies γ₁ and γ₂ are derived from the material properties of the microsphere, fluid suspension, and partially cross-linked receiving layer, and are indicative of the forces acting on the microspheres (see formulas 1 and 2). For example, in FIG. 9, when the ratio of γ₁ to γ₂ is equal to 2, the modulus of the partially cross-linked receiving layer should be between 1 MPa and 55 MPa. The results for other cases of microsphere diameter and density from Table 3 are shown in FIGS. 10-14. For practical purposes, the lower bound for the modulus can be chosen as 1 MPa. The upper and lower bounds for the modulus can be optimized depending on the number of microspheres per unit area, the microsphere radius, and the separation distance L, using the formulas provided herein.

TABLE 1

| Nearest neighbor | No. of |
|---|---|
| separation distance, μ | microspheres |
| 0-10 | 10 |
| 10-20 | 27 |
| 20-30 | 10 |
| 30-40 | 27 |
| 40-50 | 32 |
| 50-60 | 16 |
| 60-70 | 38 |
| 70-80 | 36 |
| 80-90 | 41 |
| 90-100 | 37 |
| 100-110 | 40 |
| 110-120 | 48 |
| 120-130 | 55 |
| 130-140 | 50 |
| 140-150 | 66 |
| 150-160 | 49 |
| 160-170 | 37 |
| 170-180 | 35 |
| 180-190 | 44 |
| 190-200 | 32 |
| 200-210 | 43 |
| 210-220 | 39 |
| 220-230 | 22 |
| 230-240 | 26 |
| 240-250 | 16 |
| 250-260 | 24 |
| 260-270 | 11 |
| 270-280 | 18 |
| 280-290 | 15 |
| 290-300 | 11 |
| 300-310 | 4 |
| 310-320 | 6 |
| 320-330 | 8 |
| 330-340 | 6 |
| 340-350 | 5 |
| 350-360 | 4 |
| 360-370 | 1 |
| 370-380 | 0 |
| 380-390 | 1 |
| 390-400 | 3 |
| 400-410 | 0 |
| 410-420 | 1 |
| 420-430 | 2 |
| 430-440 | 0 |
| 440-450 | 2 |
| 450-460 | 0 |
| 460-470 | 0 |
| 470-480 | 1 |
| 480-490 | 0 |
| 490-500 | 0 |
| 500-510 | 0 |
| 510-520 | 0 |
| 520-530 | 0 |
| 530-540 | 0 |
| 540-550 | 0 |
| 550-560 | 0 |
| 560-570 | 1 |
| 570-580 | 0 |
| 580-590 | 0 |
| 590-600 | 0 |
| 600-610 | 0 |
| 610-620 | 0 |
| 620-630 | 0 |
| 630-640 | 0 |
| 640-650 | 0 |
| 650-660 | 0 |
| 660-670 | 0 |
| 670-680 | 0 |
| 680-690 | 0 |
| 690-700 | 0 |
| TOTAL | 1000 |

TABLE 2

| | No. of | | | No. of | |
|---|---|---|---|---|---|
| Nearest neighbor | microsp | Cumulative | Nearest neighbor | microsp | Cumulative |
| separation distance, μ | heres | average | separation distance, μ | heres | average |
| 0-10 | 9.4 | 100 | 350-360 | 3.4 | 2.145 |
| 10-20 | 16.65 | 99.06 | 360-370 | 2.55 | 1.805 |
| 20-30 | 22.4 | 97.395 | 370-380 | 2.8 | 1.55 |
| 30-40 | 25.3 | 95.155 | 380-390 | 2.6 | 1.27 |
| 40-50 | 32.9 | 92.625 | 390-400 | 1.45 | 1.01 |
| 50-60 | 34.6 | 89.335 | 400-410 | 1.8 | 0.865 |
| 60-70 | 37.4 | 85.875 | 410-420 | 1.15 | 0.685 |
| 70-80 | 39.55 | 82.135 | 420-430 | 1.45 | 0.57 |
| 80-90 | 42.55 | 78.18 | 430-440 | 0.9 | 0.425 |
| 90-100 | 42.05 | 73.925 | 440-450 | 0.55 | 0.335 |
| 100-110 | 45.8 | 69.72 | 450-460 | 0.7 | 0.28 |
| 110-120 | 47.5 | 65.14 | 460-470 | 0.45 | 0.21 |
| 120-130 | 49.3 | 60.39 | 470-480 | 0.6 | 0.165 |
| 130-140 | 52.2 | 55.46 | 480-490 | 0.3 | 0.105 |
| 140-150 | 45.7 | 50.24 | 490-500 | 0.1 | 0.075 |
| 150-160 | 47.0 | 45.67 | 500-510 | 0.2 | 0.065 |
| 160-170 | 40.15 | 40.97 | 510-520 | 0.1 | 0.045 |
| 170-180 | 39.15 | 36.955 | 520-530 | 0.05 | 0.035 |
| 180-190 | 37.2 | 33.04 | 530-540 | 0 | 0.03 |
| 190-200 | 32.4 | 29.32 | 540-550 | 0 | 0.03 |
| 200-210 | 31.5 | 26.08 | 550-560 | 0.05 | 0.03 |
| 210-220 | 28.05 | 22.93 | 560-570 | 0.05 | 0.025 |
| 220-230 | 28.65 | 20.125 | 570-580 | 0 | 0.02 |
| 230-240 | 25.95 | 17.26 | 580-590 | 0.05 | 0.02 |
| 240-250 | 21.5 | 14.665 | 590-600 | 0 | 0.015 |
| 250-260 | 17.85 | 12.515 | 600-610 | 0.05 | 0.015 |
| 260-270 | 14.8 | 10.73 | 610-620 | 0.05 | 0.01 |
| 270-280 | 16.1 | 9.25 | 620-630 | 0 | 0.005 |
| 280-290 | 11.35 | 7.64 | 630-640 | 0 | 0.005 |
| 290-300 | 9.75 | 6.505 | 640-650 | 0 | 0.005 |
| 300-310 | 7.8 | 5.53 | 650-660 | 0 | 0.005 |
| 310-320 | 8.85 | 4.75 | 660-670 | 0 | 0.005 |
| 320-330 | 6.75 | 3.865 | 670-680 | 0.05 | 0.005 |
| 330-340 | 6.1 | 3.19 | 680-690 | 0 | 0 |
| 340-350 | 4.35 | 2.58 | 690-700 | 0 | 0 |

TABLE 3

| No. of | Microsphere | |
|---|---|---|
| microspheres/cm² | Diameter, μ | L, μ |
| 1000 | 5 | 30* |
| 1000 | 10 | 30** |
| 1000 | 15 | 20* |
| 1000 | 20 | 20* |
| 10000 | 10 | 5* |
| 10000 | 20 | 2.5* |

*96% of microspheres are separated by >L from their nearest neighbors
**95% of microspheres are separated by >L from their nearest neighbors

The invention has been described in detail with particular reference to certain embodiments thereof. Variations and modifications can be effected within the spirit and scope of the invention.

### PARTS LIST

1 support
2 fluid layer
3 microspheres (beads)
4 gel
5 microarray
6 cross-linked fluid layer
7 film of gelling agent
8 non-cross-linked gelling agent
9 receiving layer
10 partially cross-linked receiving layer
11 fluid suspension
12 chemically cross-linked receiving layer
13 radiation
14 sol-gel transitioned receiving layer

1. A method for fabricating an element comprising an array of microspheres on a support, the method comprising:
a) coating a support with a coating composition to form a receiving layer, said layer having a modulus that can be modified by crosslinking;
b) allowing partial cross-linking of the receiving layer to achieve an elastic modulus that permits partial submerging of the microspheres into the partially cross-linked receiving layer;
c) coating on the partially cross-linked receiving layer a dispersion of microspheres in a fluid suspension, each microsphere having a position;
d) allowing the microspheres to partially submerge into the partially cross-linked receiving layer;
e) removing the fluid suspension from the partially cross-linked receiving layer; and
f) allowing the partially cross-linked receiving layer to further cross-link so that the microspheres maintain their respective positions during and after wet processing.

2. The method of claim 1 wherein said microspheres form an interface with the partially cross-linked receiving layer, and the interface has an interfacial surface energy γ₁.

3. The method of claim 1 wherein said microspheres form an interface with the fluid suspension, and the interface has an interfacial surface energy γ₂.

4. The method of claim 1 wherein the modulus of said partially cross-linked receiving layer has a lower bound of 1 MPa.

5. The method of claim 1 wherein the modulus of the partially cross-linked receiving layer is defined by a monotonically increasing function of a ratio of γ₁ to γ₂, wherein γ₁ is the interfacial surface energy between the microspheres and the receiving layer and γ₂ is the interfacial surface energy between the microspheres and the fluid suspension.

6. An element comprising:
a) a support;
b) a water-insoluble receiving layer on the support, wherein the receiving layer comprises a receiving layer material; and
c) randomly-spaced microspheres fixed and partially submerged in the receiving layer, wherein the microspheres have surfaces exposed above the receiving layer, each exposed surface having at least one probe attached for interacting with an analyte,
and wherein the exposed surfaces of the microspheres are free of receiving layer material.

7. The element of claim 6 wherein the crosslinked layer contains gelatin.

8. The element of claim 6 wherein the microspheres comprise polystyrene or poly(methylmethacrylate).

9. The element of claim 6 wherein the support comprises glass paper, metal or polymer.

10. The element of claim 6 wherein the microspheres have a mean diameter of 1 to 100μ.

11. The element of claim 6 wherein the microspheres have a mean diameter of 5 to 20μ.

12. The element of claim 6 wherein the number of micropheres per cm² in the crosslinked layer is between 100 and 1,000,000.

13. The element of claim 6 wherein the number of micropheres per cm² in the crosslinked layer is between 10,000 and 100,000.

14. The element of claim 6 wherein the number of micropheres per cm² in the crosslinked layer is between 1,000 and 200,000.

15. The element of claim 6 wherein the microspheres are color-coded.

16. The element of claim 15 wherein the color code of each microsphere identifies the probe on the surface of the microsphere.

17. The element of claim 6 wherein the probe is protein or nucleic acid.

18. An element comprising an array of microspheres, wherein the element is produced by a method comprising:
a) coating a support with a coating composition to form a receiving layer, said layer having a modulus that can be modified by crosslinking;
b) allowing partial cross-linking of the receiving layer to achieve an elastic modulus that permits partial submerging of the microspheres into the partially cross-linked receiving layer;
c) coating on the partially cross-linked receiving layer a dispersion of microspheres in a fluid suspension, each microsphere having a position;
d) allowing the microspheres to partially submerge into the partially cross-linked receiving layer;
e) removing the fluid suspension from the partially cross-linked receiving layer; and
f) allowing the partially cross-linked receiving layer to further cross-link so that the microspheres maintain their respective positions during and after wet processing.

19. The element of claim 18, wherein the modulus of the partially cross-linked receiving layer allows no lateral movement of the microspheres in the partially cross-linked receiving layer during removal of the fluid suspension.
